(19) United States
(12) Patent Application Publication
(10) Pub. No.: US 2019/0076091 A1
(43) Pub. Date: Mar. 14, 2019

(54) System and Method for Monitoring a Baby's Vital Signs

(71) Applicant: Babelt Holdings LLC, Houston, TX (US)

(72) Inventor: Alvaro Ovalle, Cape Coral, FL (US)

(21) Appl. No.: 15/703,459

(22) Filed: Sep. 13, 2017

(51) Int. Cl. A61B 5/00; A61N 1/08

(52) U.S. Cl. CPC A61B 5/6831; A61B 5/002; A61B 2562/16; A61B 2503/04; A61N 1/08

(57) ABSTRACT

A wearable monitoring device and method of use is disclosed. In one embodiment, the wearable monitoring device can have a casing and a strap. The casing can have a monitoring- device hardware that can have a sensor, the sensor capable of gathering data from a baby. The casing can further comprise a monitoring device processor that receives data from the monitoring-device hardware, and stores the data on a monitoring device memory, further wherein the data comprises a device identifier and vital signs information. The casing can also comprise a communication hardware capable of sending the data to an electronic device. The strap can be attachable to a part of the body of the baby, and the strap mounts to the casing.

### BACKGROUND

This disclosure relates to a system and method for monitoring a baby's vitals.

Today, sudden infant death syndrome (SIDS) remains as one of the main causes of infant's death. SIDS is known as the sudden unexplained death of a child that is less than one year of age. It is also known that SIDS usually occurs during sleep, and that there is usually no noise produced or signs of struggle from the infant. Another frightening aspect of SIDS is that a seemingly healthy infant can still be susceptible to such syndrome. Thus, different measures and/or method is being followed to prevent this from happening. The most common methods involve traditional approaches such as putting an infant on its back when sleeping, avoiding loose blankets in the crib, using a pacifier at sleep time, and so on. Though the said methods can prevent infant's death, such methods can still be inadequate in monitoring an infant's breathing condition. Other methods include putting a baby monitor on a baby, which can include putting wearable devices on a baby to track baby's vitals. This method can be effective in monitoring the babies vital. However, monitoring devices usually comprise plastic materials that can crack or break into sharp pieces. The sharp pieces can accidentally cut a baby's skin or be lodged in the mouth of a child causing infection or possibly death. As such it would be useful to have an improved system and method for monitoring a baby's vitals.

### SUMMARY

A wearable monitoring device is disclosed. In one embodiment, the wearable monitoring device can comprise a casing and a strap. The casing can comprise a monitoring-device hardware that can comprise a sensor, the sensor capable of gathering data from a baby. The casing can further comprise a monitoring device processor that receives data from the monitoring-device hardware, and stores the data on a monitoring device memory, further wherein the data comprises a device identifier and vital signs information. The casing can also comprise a communication hardware capable of sending the data to an electronic device. The strap can be attachable to a part of the body of the baby, and the strap mounts to the casing.

A method for monitoring a baby's vital signs is disclosed. The method can comprise comprising the steps of attaching a wearable monitoring device to a baby, the wearable monitoring device as described above. The method can further comprise the steps of monitoring the vital signs information of the user through the sensor, sending the data continuously to a computing device through a network, receiving status of the vital signs information from said computing device via said network; and indicating the status through an indicator, the indicator placed on the casing.

### BRIEF DESCRIPTION OF THE DRAWINGS

FIG. 1A illustrates a wearable monitoring device.

FIG. 1B illustrates a slip-on embodiment of a wearable monitoring device.

FIG. 1C illustrates an underside exterior of a monitoring device comprising a sensor.

FIG. 2 illustrates a non-limiting circuit board embodiment of a monitoring device.

FIG. 3 illustrates internal components of a wearable monitoring-device.

FIG. 4 illustrates an electronic mobile device showing a user's vital information.

FIG. 5 illustrates internal hardware of an electronic mobile device.

FIG. 6 illustrates a vital sign monitoring system.

FIG. 7 illustrates a schematic diagram of a server.

FIG. 8 illustrates a user account.

FIG. 9 illustrates an exemplary method for monitoring a baby's vitals using a monitoring device.

### DETAILED DESCRIPTION

Described herein is a system and method for monitoring a baby's vitals. The following description is presented to enable any person skilled in the art to make and use the invention as claimed and is provided in the context of the particular examples discussed below, variations of which will be readily apparent to those skilled in the art. In the interest of clarity, not all features of an actual implementation are described in this specification. It will be appreciated that in the development of any such actual implementation (as in any development project), design decisions must be made to achieve the designers' specific goals (e.g., compliance with system- and business-related constraints), and that these goals will vary from one implementation to another. It will also be appreciated that such development effort might be complex and time-consuming, but would nevertheless be a routine undertaking for those of ordinary skill in the field of the appropriate art having the benefit of this disclosure. Accordingly, the claims appended hereto are not intended to be limited by the disclosed embodiments, but are to be accorded their widest scope consistent with the principles and features disclosed herein.

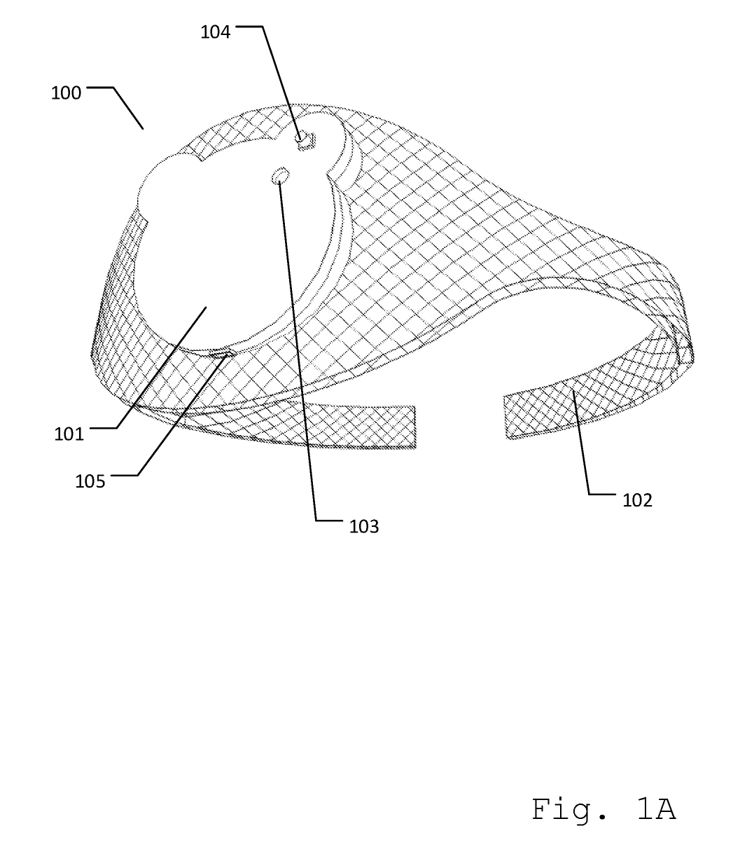

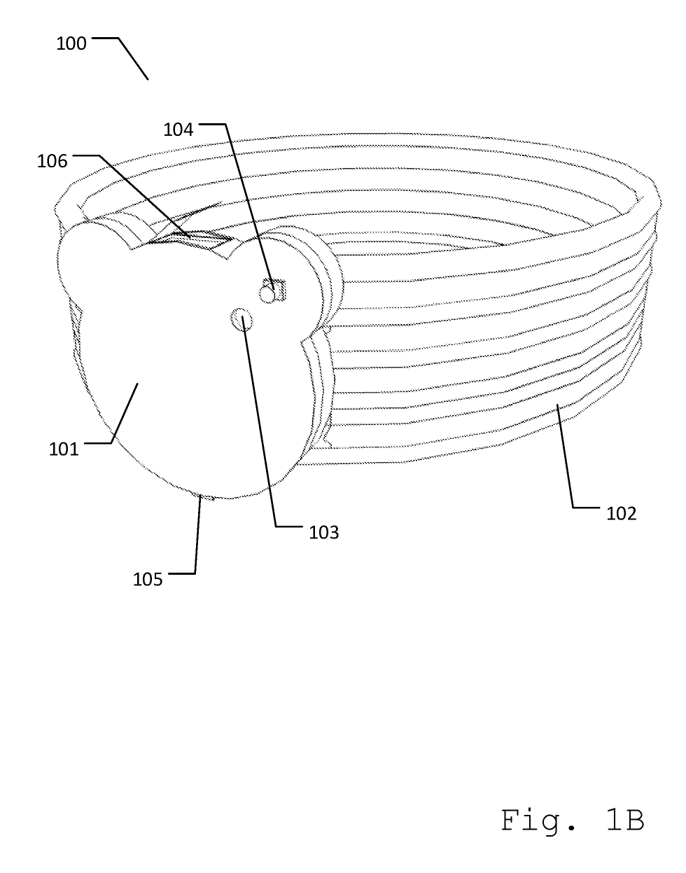

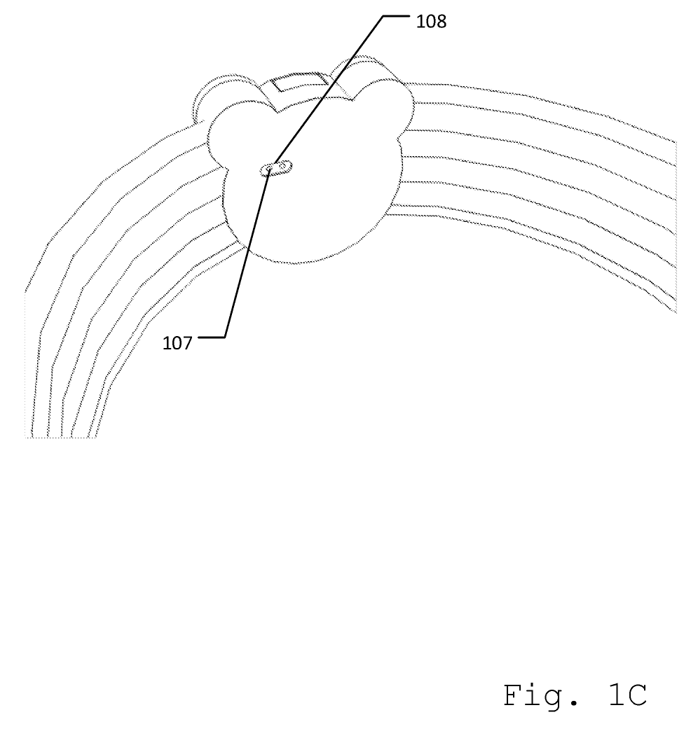

FIG. 1A illustrates a wearable monitoring device 100. Wearable monitoring device 100 can be any piece of object or accessories that can be worn and used to monitor vital signs of a baby. For purposes of this disclosure, vital signs that can be measured for an infant can include but are not limited to heart rate, and body temperature. Moreover, wearable monitoring device 100 can monitor noise surrounding the user which can indicate if the user is sleeping well, awake, or is being disturbed with unnecessary noise. Wearable monitoring device 100 can include but are not limited to socks, bracelets, and watch that can be worn to monitor the user's vital signs. In a preferred embodiment, wearable monitoring device 100 can be in a form of a bracelet as shown in FIG. 1. In this embodiment, wearable monitoring device 100 can comprise a casing 101 and a strap 102. Casing 101 can be an enclosure capable of housing and securing internal parts of wearable monitoring device 100. Casing 101 can be made of durable material such as a rubber and can have variety of designs, shapes, and/or sizes. Strap 102 can adaptably wrap around the wrist of the baby. Moreover, strap 102 can be the predominant portion of wearable monitoring device 100 that can mount casing 101. In one embodiment, strap 102 can comprise of durable materials that can include but is not limited to rubber, and/or fabric materials. In a preferred embodiment, strap 102 can be made of a toxic non-hypoallergenic, and soft material such as cotton. In one embodiment, casing 101 can attach to strap 102 through method that can include but is not limited to adhesion, sewing, or fastening. In such embodiment, casing 101 and strap 102 can be a separate device. In another embodiment, casing 101 and strap 102 can be a unibody. Further, casing 101 can comprise an indicator 103, a reset button 104, and a charging port 105. Indicator 103 can comprise light-emitting diode (LED). Indicator 103 can indicate that wearable monitoring device 100 is powered on. In one embodiment, indicator 103 can use different colors to indicate status of the user's vitals. In another embodiment, indicator 103 can use blinking lights to indicate status of the baby's vitals. Reset button 104 can be placed at the exterior surface of casing 101. Reset button 104 can be actuated when there is a malfunction in the operations of monitoring device 100. Charging port 105 can be used to recharge the battery of monitoring device 100. Charging port 105 can be any type of standard mobile device charger, which can include but is not limited to a lightning port, or a USB (Universal Serial Bus) port. Some examples of USB port can include micro USB port, mini USB port, and Type A port.

FIG. 1B illustrates a slip-on embodiment of wearable monitoring device 100. In such embodiment, strap 102 can be made of slip-on fabric. Examples of slip-on fabric can include but are not limited to rubberized fabric, or elastic bands. Moreover, strap 102 can comprise a smooth exterior, to ensure that the baby can barely feel the device. Thus, irritability or discomfort on the baby can be prevented and markings on the baby's arm can be reduced. Further in one embodiment, monitoring device 100 can comprise a battery 106. Battery 106 can provide power for the operation of monitoring device 100. Moreover, battery 106 can be placed within casing 101. This feature ensures that battery 106 is away from baby and not in contact with the baby's skin. In one embodiment, battery 106 can be removable from casing 101. An example of, battery 106 can be a lithium-ion (li-ion) rechargeable battery. In another embodiment, battery 106 can be removable from casing 101. In such embodiment, battery 106 can be replaced with a new battery.

FIG. 1C illustrates an underside exterior of monitoring device 100 comprising a sensor 107. As a non-limiting embodiment, sensor 107 can be a laser. Sensor 107 can be mounted within casing 101 and can be capable of scanning and monitoring user's vitals. In such embodiment, strap 102 can comprise an opening 108 to allow a portion of sensor 107 be in contact with the baby's skin.

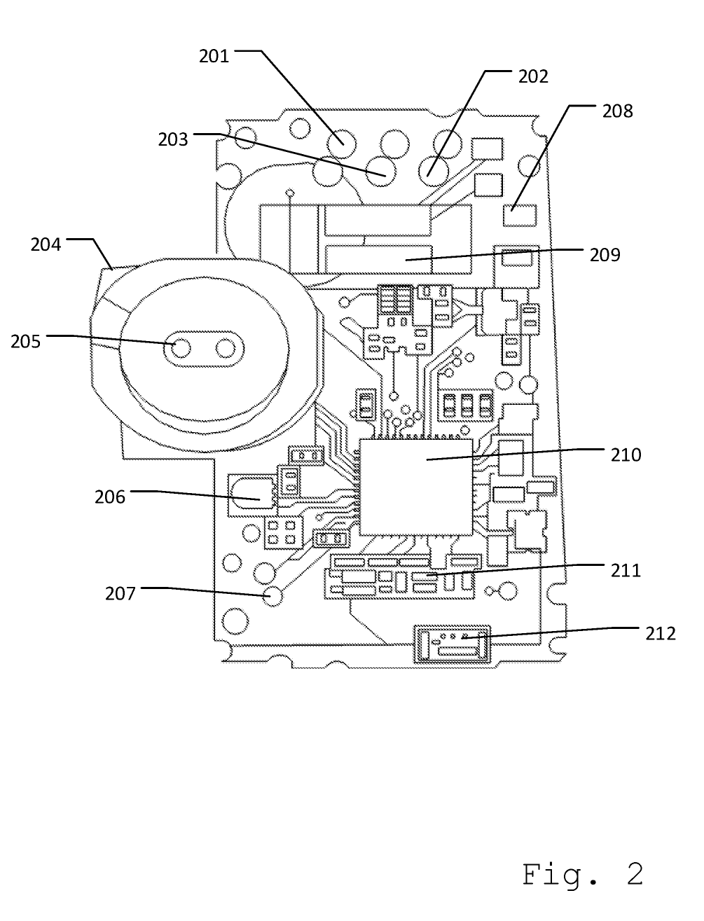

FIG. 2 illustrates a non-limiting circuit board 200 embodiment of monitoring device 100. Circuit board 200 can comprise a plurality of gold connectors. 201-203. The gold connectors can be 5V DC in one embodiment. capacity heart rate sensor 204 comprising double laser lenses 205, a blood pressure chip 206, a SWSLK-130 207, one or more Bluetooth communication chips 208, 212, a heart rate chip 209, a microprocessor 210, and high connectivity sensors 211.

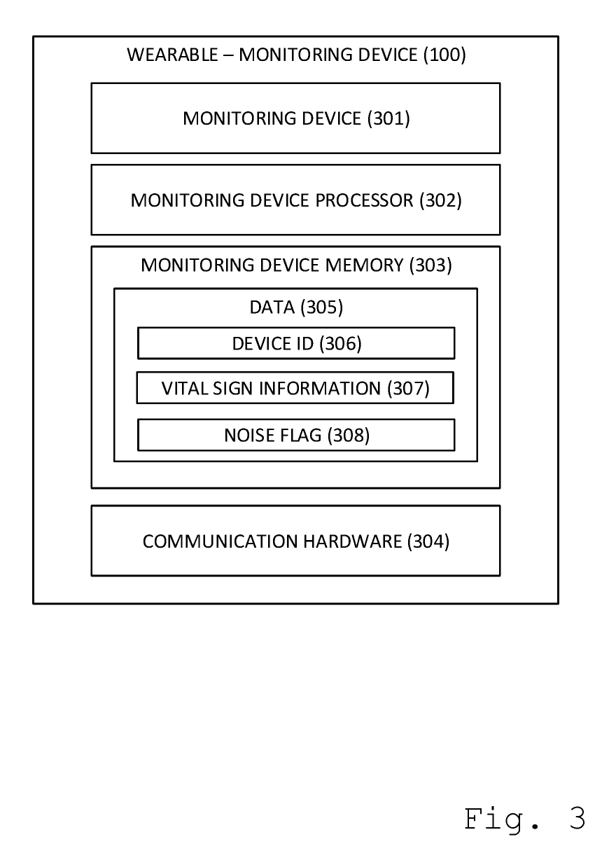

FIG. 3 illustrates internal components of wearable monitoring-device 100 comprising a monitoring-device hardware 301, a monitoring device processor 302 such as microprocessor 210, a monitoring device memory 303, and a communication hardware 304. Monitoring-device hardware 301 can comprise different kinds of sensor 107 capable of monitoring the baby's vital status. Sensor 107 can include but is not limited to biosensor, temperature sensor, and/or noise monitoring sensor. As an exemplary embodiment, monitoring-device hardware 301 can include double laser lenses 205. Data 305 can be collected from monitoring-device hardware 301 and can be sent to monitoring-device processor 302. In one embodiment, data 305 can include a device identifier (ID) 306, a vital signs information 307, and a noise flag 308. Device ID 306 can be a unique identification of wearable monitoring-device 100, which can comprise of alphanumeric characters. Vital signs information 307 can include but is not limited to body temperature, heart rate, and child's sleeping condition. Noise flag 308 can be a flag that indicates the noise condition around monitoring device 100. As such, when noise can be detected within the area of monitoring device 100 noise flag 308 can be flagged or marked. Monitoring-device processor 302 can be a device that performs executable functions on monitoring-device memory 303. In one embodiment, monitoring-device processor 302 can send instructions to indicator 103. As such, indicator 103 can light up and/or operate according to the instructions sent by monitoring-device processor 302. Monitoring-device memory 303 can be a physical device used to store programs and/or data. Processes of monitoring-device processor 302 can include storing data 305 to monitoring-device memory 303, and verifying data 305 is valid and conforms to preset standards. Communication hardware 304 can include a network transport processor for packetizing data, communication ports for wired communication, or an antenna for wireless communication. Further, data 305 can be sent to communication hardware 304 for communication over a network. In one embodiment, communication hardware 304 can allow transmitting and receiving communication with an electronic device via Bluetooth. In another embodiment, communication hardware 304 can allow wearable monitoring-device 100 to transmit and/or receive data to an electronic device through a wireless network connection.

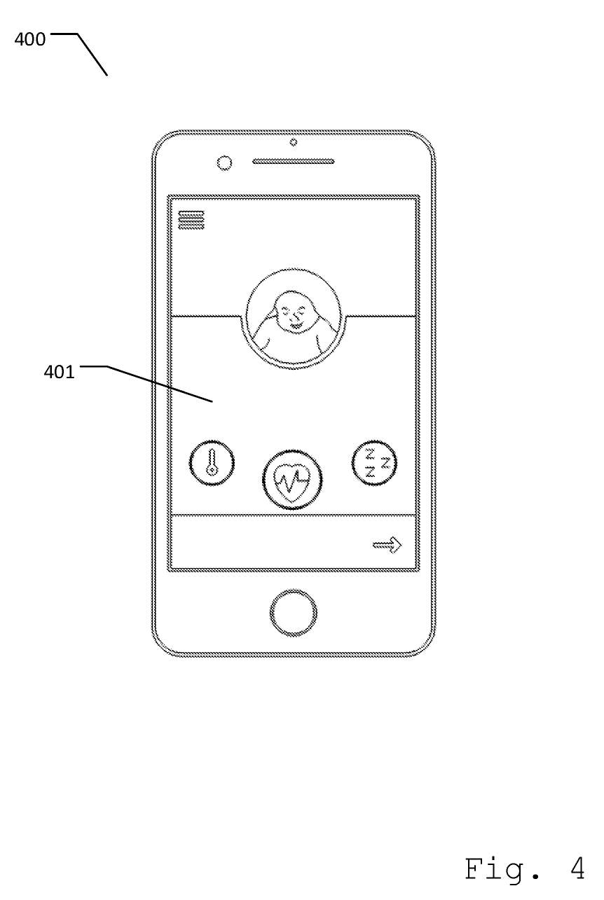

FIG. 4 illustrates an electronic mobile device 400 showing user's vital information 307. Electronic mobile device 400 can include, but is not limited to, a mobile phone, a laptop, a personal digital assistant (PDA), and/or a tablet. Electronic mobile device 400 can comprise a screen 401. In one embodiment, electronic mobile device 400 can be programmed essentially for the function of displaying, receiving, and collecting information from wearable monitoring-device 100 through a network. In another embodiment, an application can be installed to an electronic mobile device 400 to perform the function of displaying, receiving, and collecting information from wearable monitoring-device 100. In such embodiments, electronic mobile device 400 can display data 305 through screen 401. As an example, a user information can be shown on screen 401, which can include but is not limited to a child's profile such as name, picture, and/or age, a child's temperature, a child's heart rate, and a child's sleeping condition.

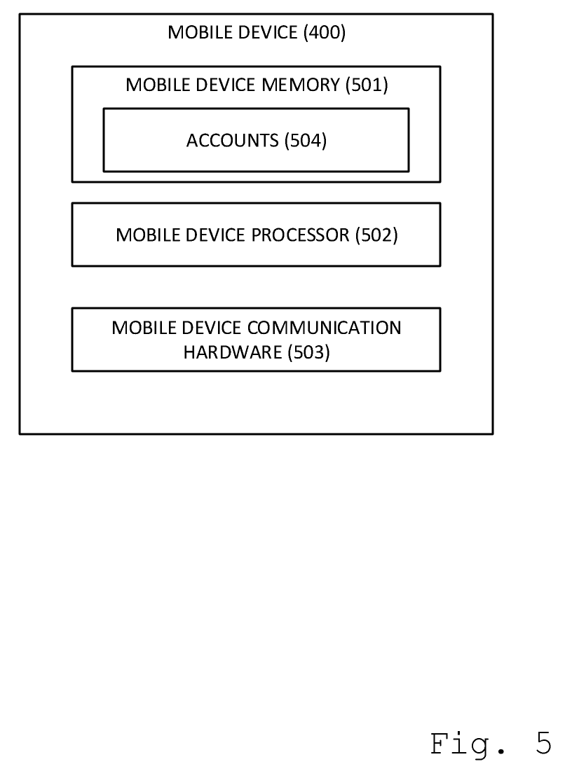

FIG. 5 illustrates internal hardware of electronic mobile device 400 comprising a mobile device memory 501, a mobile device processor 502, and mobile device communication hardware 503. Data 305 from wearable monitoring-device 100 can be sent to electronic mobile device processor 502. Mobile device processor 502 can perform processes on the data according to an application stored in mobile device memory 501, as discussed further below. Processes can include storing collected data 305 to mobile device memory 501, verifying collected data 305 conforms to preset standards or ensuring all required data has been gathered for information inquiry to be complete. Mobile device memory 501 can comprise one or more accounts 504.

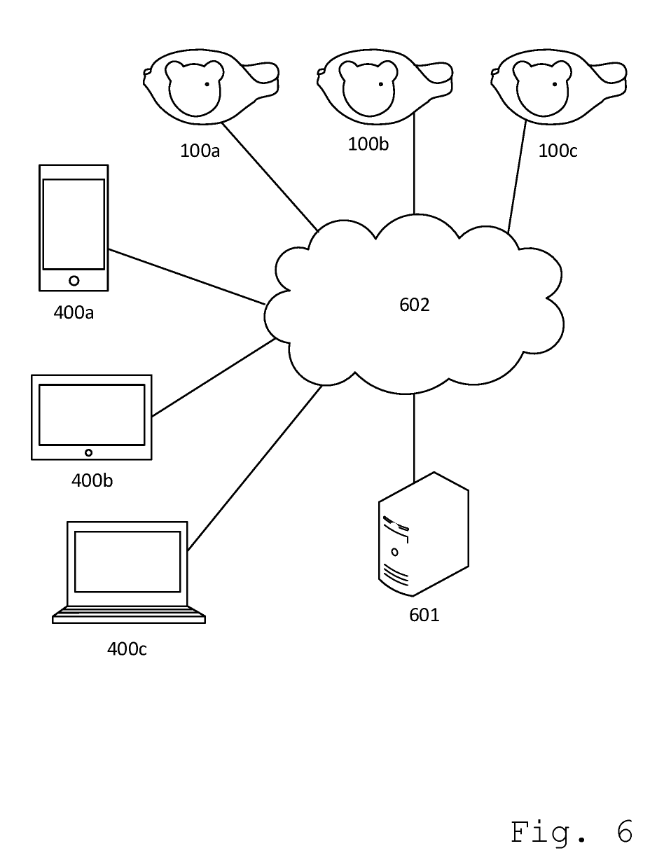

FIG. 6 illustrates a vital sign monitoring system comprising one or more wearable monitoring devices 100, one or more electronic mobile devices 400, and one or more servers 601 connected via a network 602. Server 601 can represent at least one, but can be many servers, each connected to network 602. Server 601 can be accessible to an individual or a group of individuals through a web browser application. Network 602 can be a local area network (LAN), a wide area network (WAN), a piconet, or a combination of LANs, WANs, or piconets. One illustrative LAN is a network within a single business. One illustrative WAN is the Internet. In the preferred embodiment, network 602 will comprise the Internet.

In one embodiment, data 305 from each monitoring device 100 can communicate with electronic mobile devices 400 through network 602. In such embodiment, data 305 can be sent from each monitoring device 100 to each corresponding mobile device 400 via a Bluetooth. In another embodiment, data 305 can be sent from each monitoring device 100 to each corresponding mobile device 400 via a WIFI (wireless fidelity) connection. Further in another embodiment, each monitoring device 100 can send data 305 to server 601 through WIFI. In such embodiment, server 601 can receive and transfer data 305 to each corresponding mobile device 400. In this embodiment, a first user can wear a first monitoring device 100a of one or more wearable monitoring devices 100, a second user can wear a second monitoring device 100b, and so on. In such example embodiment, data 305 can be sent from first monitoring device 100a to a first mobile device 400a through network 602.

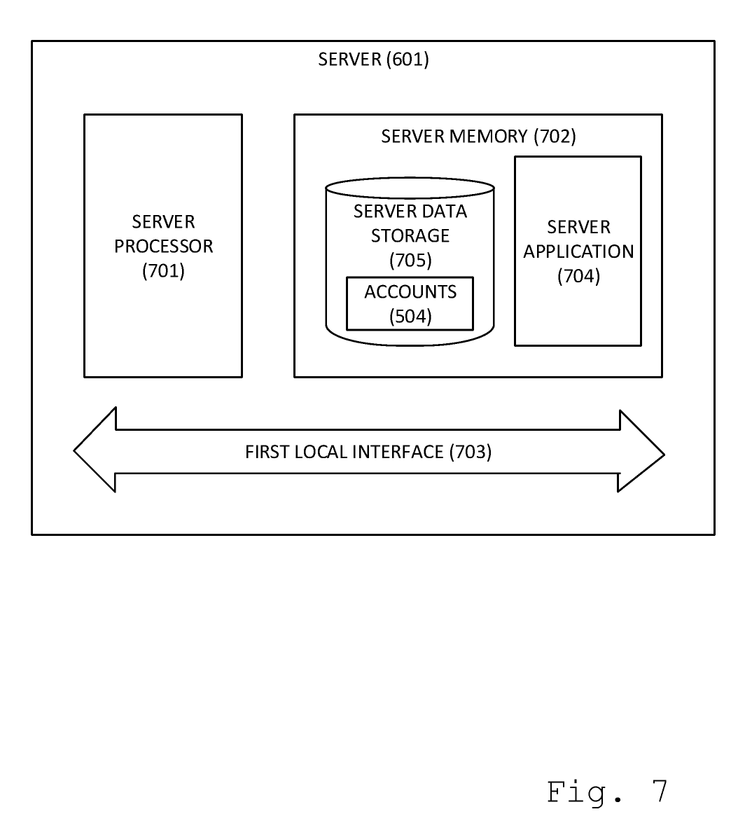

FIG. 7 illustrates a schematic diagram of server 601 according to an embodiment of the present disclosure. Server 601 can comprise a server processor 701, a server memory 702, and a first local interface 703. Server 601 can comprise a server processor 701, and a first local interface 703. First local interface 703 can be a program that controls a display for the user, which can allow user to view and/or interact with server 601. Server 601 can be a processing unit that performs a set of instructions stored within server memory 702. Server memory 702 can comprise a server application 604, and a server data store 705. Server application 704 can be a program providing logic for server 601. Server data store 705 can be collections of data accessible through server application 704. Server data store 705 can comprise accounts 504. Further, server application 704 can perform functions such as adding, transferring, and retrieving information on server data store 705 using first local interface 703.

Server 601 includes at least one processor circuit, for example, having server processor 701 and server memory 702, both of which are coupled to first local interface 703. To this end, the server 601 can comprise, for example, at least one server, computer or like device. First local interface 703 can comprise, for example, a data bus with an accompanying address/control bus or other bus structure as can be appreciated.

Both data and several components that are executable by server processor 701 are stored in server memory 702. In particular, server application 704 and, potentially, other applications are stored in the server memory 702 and executable by server processor 701. Also, server data store 705 and other data can be stored in server memory 702. In addition, an operating system can be stored in server memory 702 and executable by server processor 701.

Further, it is understood that there are other applications that can be stored in mobile device memory 501 and server memory 702 and executable by mobile device processor 502 and server processor 701. Where any component discussed herein is implemented in the form of software, any one of a number of programming languages can be employed such as, for example, C, C++, C#, Objective C, Java, Java Script, Perl, PHP, Visual Basic, Python, Ruby, Delphi, Flash, or other programming languages.

A number of software components can be stored in mobile device memory 501 and server memory 702 and can be executable by mobile device processor 502 and server processor 701. In this respect, the term “executable” can mean a program file that is in a form that can ultimately be run by mobile device processor 502 and server processor 701. Examples of executable programs can include a compiled program that can be translated into machine code in a format that can be loaded into a random access portion of mobile device memory 501 and server memory 702 and run by mobile device processor 502 and server processor 701, source code that can be expressed in proper format such as object code that is capable of being loaded into a random access portion of mobile device memory 501 and server memory 702 and executed by mobile device processor 502 and server processor 701, or source code that can be interpreted by another executable program to generate instructions in a random access portion of mobile device memory 501 and server memory 702 to be executed by mobile device processor 502 and server processor 701, etc. An executable program can be stored in any portion or component of mobile device memory 501 and server memory 702 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

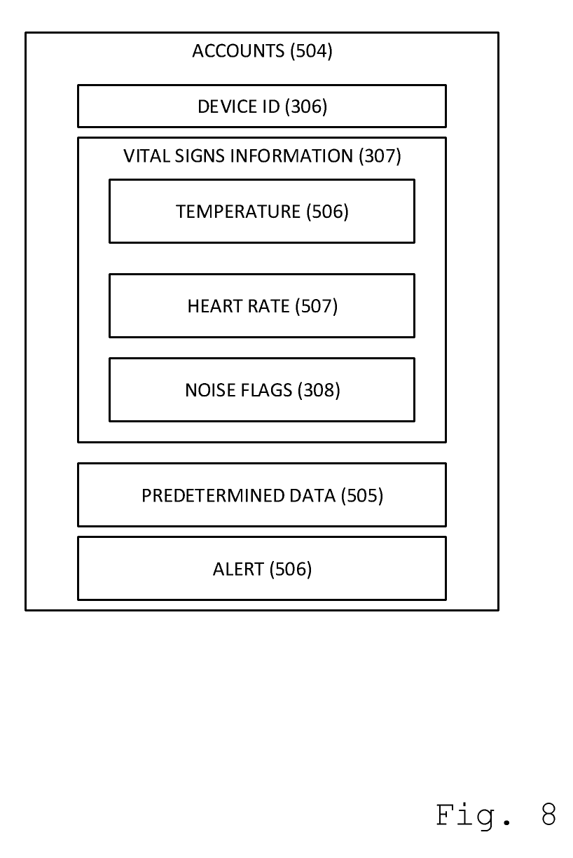

FIG. 8 illustrates accounts 504 can comprise device ID 306, vital signs information 307, predetermined data 505, and alert 506. Accounts 504 can comprise information such as a child's name, sex, age, date of birth, and weight. Vital signs information 307 can comprise a real-time reading of a child's temperature 506, a child's heart rate 507, and noise flag 308. Predetermined data 505 can be predetermined information of a child's normal vital signs that is according to age, time of the day, and/or weather condition. In one embodiment, alert 506 can be a sound notification. In another embodiment, alert 506 can be a message notification.

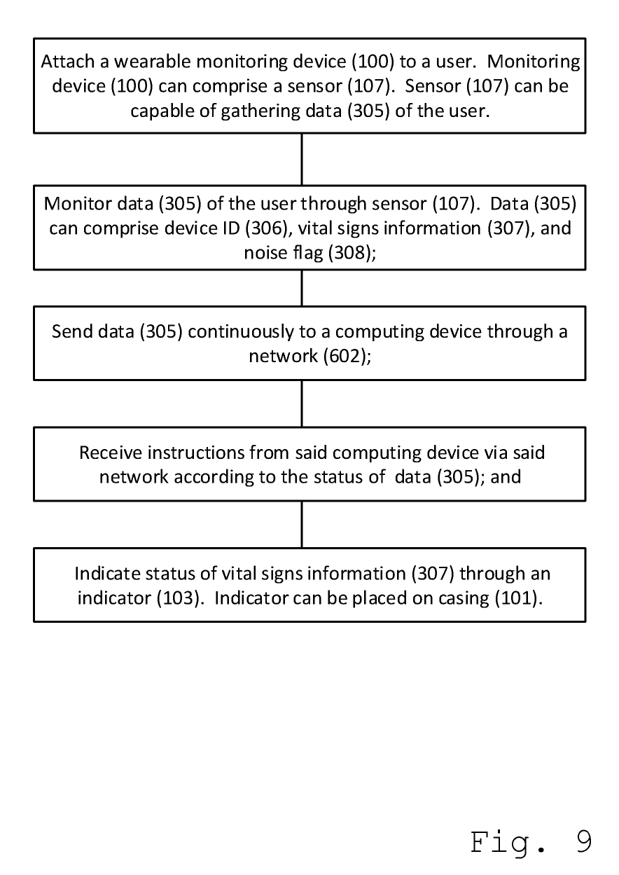

FIG. 9 illustrates an exemplary method for monitoring a user's vitals using monitoring device 100. First, monitoring device 100 can be worn by or attached to a user. Once attached, sensor 107 can start monitoring vital signs information 307 of the user. Data 305 gathered through monitoring device 100 can be sent continuously to a computing device through network 602. For purposes of this disclosure, computing device can be any machine capable of performing calculations automatically. A non-limiting example of computing device can comprise electronic device, 400, and server 601.

Computing device continuously receives data 305 in real time. As such, a computing device processor can compare vital signs information 307 with predetermined data 505. In scenario wherein one or more of user's vital signs information 307 can be outside the range of predetermined data 505, alert 506 can be triggered on the computing device. Further in another scenario wherein noise can be detected by monitoring device 100, noise flag 308 can be flagged that can indicate to the computing device that noise was detected at the location of monitoring device 100. This can also indicate that the user is awake, or that there are noises around the user that can cause user to wake up. In such scenario, alert 506 can be triggered. In an embodiment, wherein the computing device is server 601, server 601 can send instructions to electronic device 400 to indicate that alert 506 was triggered. Thus, alert notification can be displayed and/or sound notification can be played on electronic device 400. In another embodiment, wherein the computing device is electronic device 400, electronic device 400 can trigger alert 506. Accordingly, the computing device can send instructions to monitoring device 100. As such, indicator 103 on monitoring device 100 can operate according to the instructions received from the computing device. In a scenario, wherein vital signs information 307 can be within the range of predetermined data 505, vital signs information 307 can be continuously monitored and alert 506 cannot be triggered. As an exemplary embodiment, when alert 506 was not triggered on the computing device, a green light on indicator 103 can be shown on monitoring device 100. This can indicate that vital signs on the user are normal. As another example, when alert 506 were triggered on the computing device, a red light on indicator 103 can be displayed on monitoring device 100. This can indicate that there's a problem on baby's vitals. In some embodiments, indicator 103 can use blinking lights to indicate a problem in a baby's vital. As an example, a steady light on indicator 103 can indicate that a problem has been detected with a baby's vital signs, and a blinking light on indicator 103 can indicate that vitals of the user are normal.

Mobile device memory 501 and server memory 702 can include both volatile and non- volatile memory and data storage components. Volatile components do not retain data values upon loss of power. Non-volatile components, on the other hand, retain data upon a loss of power. Thus, mobile device memory 501 and server memory 702 can comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM can comprise, for example, static random-access memory (SRAM), dynamic random access memory (DRAM), or magnetic random access memory (MRAM) and other such devices. The ROM can comprise, for example, a programmable read-only memory (PROM), an erasable programmable read-only memory (EPROM), an electrically erasable programmable read-only memory (EEPROM), or other like memory device.

Also, mobile device processor 502 and server processor 701 can represent multiple processors. Likewise, mobile device processor 502 and server processor 701 can represent multiple memories that operate in parallel processing circuits, respectively. In such a case, first local interface 703 can be an appropriate network, including network 602 that facilitates communication between any two of the mobile device processor 502 and server processor 701, between any mobile device processor 502 and server processor 701 and any of the mobile device memory 501 and server memory 702, or between any two of the mobile device memory 501 and server memory 702, etc. First local interface 703 can comprise additional systems designed to coordinate this communication, including, but not limited to, performing load balancing. Mobile device processor 502 and server processor 701 can be of electrical or of some other available construction.

Although server application 704, and other various systems described herein can be embodied in software or code executed by general purpose hardware discussed above, server application 704 can also be embodied in dedicated hardware or a combination of software/general purpose hardware and dedicated hardware. If embodied in dedicated hardware, each server application 704 can be implemented as a circuit or state machine that employs a number of technologies. These technologies can include, but are not limited to, discrete logic circuits having logic gates for implementing various logic functions upon an application of one or more data signals, application specific integrated circuits having appropriate logic gates, or other components, etc. Such technologies are generally well known by those skilled in the art and, consequently, are not described in detail herein.

The flowchart of FIG. 9 shows the functionality and operation of an implementation of portions of server application 704. If embodied in software, each block can represent a module, segment, or portion of code that comprises program instructions to implement the specified logical function(s). The program instructions can be embodied in the form of source code that comprises human-readable statements written in a programming language or machine code that comprises numerical instructions recognizable by a suitable execution system such as mobile device processor 502 in a computer system or other system. The machine code can be converted from the source code, etc. If embodied in hardware, each block can represent a circuit or a number of interconnected circuits to implement the specified logical function(s).

Although the flowchart of FIG. 9 show a specific order of execution, the order of execution can differ from what is depicted. For example, the order of execution of two or more blocks can be rearranged relative to the order shown. Also, two or more blocks shown in succession in flowchart of FIG. 9 can be executed concurrently or with partial concurrence. In addition, any number of counters, state variables, warning semaphores, or messages might be added to the logical flow described herein, for purposes of enhanced utility, accounting, performance measurement, or providing troubleshooting aids, etc. All such variations are within the scope of the present disclosure.

Also, any logic or application described herein that comprises software or code, including server application 704, can be embodied in any computer-readable storage medium for use by or in connection with an instruction execution system such as, mobile device processor 502 and server processor 701 in a computer system or other system. The logic can comprise statements including instructions and declarations that can be fetched from the computer-readable storage medium and executed by the instruction execution system.

In the context of the present disclosure, a “computer-readable storage medium” can be any medium that can contain, store, or maintain the logic or application described herein for use by or in connection with the instruction execution system. The computer-readable storage medium can comprise any one of many physical media, such as electronic, magnetic, optical, electromagnetic, infrared, or semiconductor media. More specific examples of a suitable computer-readable storage medium can include, but are not limited to, magnetic tapes, magnetic floppy diskettes, magnetic hard drives, memory cards, solid-state drives, USB flash drives, or optical discs. Also, the computer-readable storage medium can be a random-access memory (RAM), including static random-access memory (SRAM), dynamic random-access memory (DRAM) or magnetic random-access memory (MRAM). In addition, the computer-readable storage medium can be a read-only memory (ROM), a programmable read-only memory (PROM), an erasable programmable read-only memory (EPROM), an electrically erasable programmable read-only memory (EEPROM), or other type of memory device.

It should be emphasized that the above-described embodiments of the present disclosure are merely possible examples of implementations set forth for a clear understanding of the principles of the disclosure. Many variations and modifications can be made to the above-described embodiment(s) without departing substantially from the spirit and principles of the disclosure. All such modifications and variations are intended to be included herein within the scope of this disclosure and protected by the following claims.

Various changes in the details of the illustrated operational methods are possible without departing from the scope of the following claims. Some embodiments may combine the activities described herein as being separate steps. Similarly, one or more of the described steps may be omitted, depending upon the specific operational environment the method is being implemented in. It is to be understood that the above description is intended to be illustrative, and not restrictive. For example, the above-described embodiments may be used in combination with each other. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.”

1. A wearable monitoring device comprising
a casing that encloses
a monitoring-device hardware that comprises a sensor, said sensor capable of gathering data of a user;
a monitoring device processor that receives said data from said monitoring-device hardware, and stores said data on a monitoring device memory, further wherein said data comprises a device identifier and a vital signs information; and
a communication hardware capable of sending said data to an electronic device; and
a strap attachable to a part of the body of said user, further wherein said strap mounts said casing.

2. The wearable monitoring device of claim 1, further comprising an indicator that indicates status of said data, further wherein said monitoring device processor sends instructions to said indicator according to said data gathered from said monitoring-device hardware.

3. The wearable monitoring device monitoring device of claim 1, further comprising a reset button capable of resetting operations of said wearable monitoring device.

4. The wearable monitoring device monitoring device of claim 1, further comprising a battery and a charging port, said battery and said charging port mounted on said casing.

5. The wearable monitoring device of claim 1, wherein said sensor comprises a biosensor.

6. The wearable monitoring device of claim 1, wherein said sensor comprises a temperature sensor.

7. The wearable monitoring device of claim 1, wherein said sensor comprises a noise- monitoring sensor.

8. The wearable monitoring device of claim 1, wherein said data gathered comprises body temperature.

9. The wearable monitoring device of claim 1, said data gathered comprises heart rate.

10. The wearable monitoring device of claim 1, wherein said data gathered comprises noise condition.

11. A method for monitoring a user's vitals comprising the steps of
attaching a wearable device to a user, said wearable device comprising
a casing that encloses
a monitoring-device hardware that comprises a sensor, said sensor capable of gathering data of a user;
a monitoring device processor that receives said data from said monitoring-device hardware, and stores said data on a monitoring device memory, further wherein said data comprises a device identifier and a vital signs information; and
a communication hardware capable of sending said data to an electronic device; and
a strap attachable to a part of the body of said user, further wherein said strap mounts said casing.
monitoring said vital signs information of said user through said sensor;
sending said data continuously to a computing device through a network;
receiving status of said vital signs information from said computing device via said network; and
indicating said status through an indicator, said indicator placed on said casing.

12. The method of claim 11 further comprising the steps of
comparing said vital signs information with a predetermined data by said computing device; and
sending said status by said computing device to said wearable monitoring device through said network.

13. The method of claim 12 further comprising the step of triggering an alert on said computing device when said vital signs information is outside the range of said predetermined data.

14. The method of claim 12 wherein said alert is a sound notification.

15. The method of claim 12 wherein said alert is a message notification.

16. The method of claim 12 wherein said wearable device further comprises a reset button, said reset button capable of resetting operations of said wearable device.

17. The method of claim 11 wherein said indicator comprises light-emitting diode LED lights.

18. The method of claim 11 wherein said computing device comprises an electronic device.

19. The method of claim 11 wherein said computing device comprises a server.
